(19) United States
(12) Patent Application Publication
(10) Pub. No.: US 2021/0182568 A1
(43) Pub. Date: Jun. 17, 2021

(54) METHODS FOR IMPROVED OPERATIVE SURGICAL REPORT GENERATION USING MACHINE LEARNING AND DEVICES THEREOF

(71) Applicant: CHEMIMAGE CORPORATION, Pittsburgh, PA (US)

(72) Inventors: Jihang WANG, Sewickley, PA (US); Patrick J. TREADO, Pittsburgh, PA (US); Jeffrey K. COHEN, Pittsburgh, PA (US)

(21) Appl. No.: 17/121,099

(22) Filed: Dec. 14, 2020

Related U.S. Application Data
(60) Provisional application No. 62/947,902, filed on Dec. 13, 2019.

(51) Int. Cl. G06K 9/00; G06N 3/08; G06T 7/246; G06K 9/62; G16H 15/00; G16H 30/40; G16H 50/70; G16H 70/20; G16H 40/20; A61B 90/00; A61B 34/20

(52) U.S. Cl. CPC G06K 9/00751; A61B 2034/2065; G06K 9/00718; G06T 7/246; G06K 9/6201; G06K 9/6217; G16H 15/00; G16H 30/40; G16H 50/70; G16H 70/20; G16H 40/20; A61B 90/361; A61B 34/20; G06T 2207/10016; G06K 2209/05; G06T 2207/20081; G06T 2207/20084; G06K 2209/057; A61B 2034/2057; G06N 3/08

(57) ABSTRACT

Methods, non-transitory computer readable media, and surgical video analysis devices are disclosed that provide an improved, automated surgical report generation. With this technology, a video associated with a surgical procedure comprising a plurality of frames is obtained. The plurality of frames of the obtained video are compared to a historical set of surgical procedure images, wherein the historical set of surgical procedure images are associated with contextual information. One or more objects of interest are identified in at least a subset of the plurality of frames based on the comparison and the associated contextual information. The identified one or more objects of interest are tracked across the at least the subset of the plurality of frames. A surgical report based on tracked one or more objects.

### CROSS-REFERENCE TO RELATED APPLICATION

This application claims benefit of U.S. Provisional Patent Application No. 62/947,902, filed Dec. 13, 2019, which is hereby incorporated by reference herein in its entirety.

### FIELD OF THE DISCLOSURE

An operative report is a report written in a patient's medical record to document the details of a surgery, which must be completed immediately after an operation by surgeons. An operative report is a mandatory document required following all surgical procedures. The report has two key medical purposes: (1) to document if the procedure was completed; and (2) to provide an accurate and descriptive report of the details of the procedure. However, accurate operative reports are extremely uncommon as frequently crucial information is not transferred, placing the patient at risk for intra-operative complications.

Operative reports are also time consuming, since they are often dictated or written after the surgical procedure. In just a few hours, the surgeon has lost the major details of this particular surgery and reverts to the most common version of the report he or she uses. Operative reports are generated by dictation, or more commonly now, in written form. The surgeon often uses a template and then fills in the information, representing the current operation. In addition, a surgeon may do four of the same procedures in a row, without time in between to document each operation. Therefore operative reports, though they have a common outline known to all surgeons, vary in level of detail and are often reduced to useless information.

As such, there is a need to generate operative reports in a more accurate and efficient manner.

### SUMMARY

One aspect of the present technology relates to a method for improved, automated surgical report generation. The method includes obtaining, by a surgical video analysis device, a video associated with a surgical procedure comprising a plurality of frames. The plurality of frames of the obtained video are comparted to a historical set of surgical procedure images that are associated with contextual information. One or more objects of interest in at least a subset of the plurality of frames are identified based on the comparison and the associated contextual information. The identified one or more objects of interest are tracked across the at least the subset of the plurality of frames. A surgical report is generated based on tracked one or more objects.

Another aspect of the present invention relates to a surgical video analysis device, comprising memory comprising programmed instructions stored thereon and one or more processors configured to execute the stored programmed instructions to obtain a video associated with a surgical procedure comprising a plurality of frames. The plurality of frames of the obtained video are comparted to a historical set of surgical procedure images that are associated with contextual information. One or more objects of interest in at least a subset of the plurality of frames are identified based on the comparison and the associated contextual information. The identified one or more objects of interest are tracked across the at least the subset of the plurality of frames. A surgical report is generated based on tracked one or more objects.

A further aspect of the present invention relates to a non-transitory machine readable medium having stored thereon instructions for improved, automated surgical report generation comprising executable code that, when executed by one or more processors, causes the processors to obtain a video associated with a surgical procedure comprising a plurality of frames. The plurality of frames of the obtained video are comparted to a historical set of surgical procedure images that are associated with contextual information. One or more objects of interest in at least a subset of the plurality of frames are identified based on the comparison and the associated contextual information. The identified one or more objects of interest are tracked across the at least the subset of the plurality of frames. A surgical report is generated based on tracked one or more objects.

This technology has a number of associated advantages including providing methods, non-transitory computer readable media, and surgical video analysis devices that facilitate improved, automated operative surgical report generation. This technology automatically analyzes video(s) of a surgical procedure and generates a surgical report without requiring any intervention from the surgeon. This technology utilizes video analysis and machine learning to advantageously identify and track multiple objects in the video of the surgical procedure. The information obtained can then be analyzed, interpreted, and reported automatically on a final operative report. The analyzed data can be used in other purposes include providing references to the following surgeons of the same patient, evaluating the surgeon's performance, or contributing to clinical research. All of these advantages can potentially lower the global cost of health care, which will benefit both the patients and hospital.

### BRIEF DESCRIPTION OF THE DRAWINGS

The accompanying drawings, which are incorporated in and form a part of the specification, illustrate the embodiments of the invention and together with the written description serve to explain the principles, characteristics, and features of the invention. In the drawings:

FIG. 1 a block diagram of a network environment with an exemplary surgical video analysis device;

FIG. 2 is a block diagram of the exemplary surgical video analysis device of FIG. 1;

FIG. 3 is a flowchart of an exemplary method for improved, automated surgical report generation.

FIG. 4 is a graph of testing performance of an exemplary embodiment.

### DETAILED DESCRIPTION

This disclosure is not limited to the particular systems, methods, and non-transitory computer program products described, as these may vary. The terminology used in the description is for the purpose of describing the particular versions or embodiments only, and is not intended to limit the scope.

As used in this document, the singular forms “a,” “an,” and “the” include plural references unless the context clearly dictates otherwise. Unless defined otherwise, all technical and scientific terms used herein have the same meanings as commonly understood by one of ordinary skill in the art. Nothing in this disclosure is to be construed as an admission that the embodiments described in this disclosure are not entitled to antedate such disclosure by virtue of prior invention. As used in this document, the term “comprising” means “including, but not limited to.”

The embodiments described below are not intended to be exhaustive or to limit the teachings to the precise forms disclosed in the following detailed description. Rather, the embodiments are chosen and described so that others skilled in the art may appreciate and understand the principles and practices of the present teachings.

The disclosure contemplates systems, methods, and non-transitory computer program products that provide an improved, automated surgical report generation. With this technology, a video associated with a surgical procedure comprising a plurality of frames is obtained. The plurality of frames of the obtained video are compared to a historical set of surgical procedure images, wherein the historical set of surgical procedure images are associated with contextual information. One or more objects of interest are identified in at least a subset of the plurality of frames based on the comparison and the associated contextual information. The identified one or more objects of interest are tracked across the at least the subset of the plurality of frames. A surgical report based on tracked one or more objects.

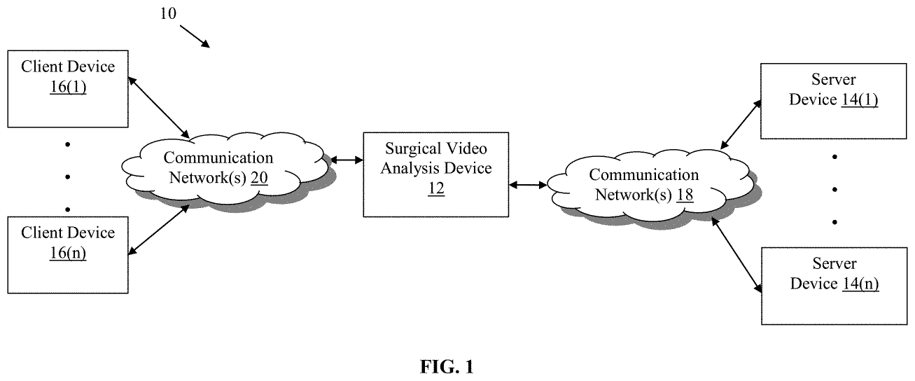

Referring to FIG. 1, an exemplary network environment 10 with an exemplary surgical video analysis device 12 is illustrated. The surgical video analysis device 12 in this example is coupled to a plurality of server devices 14(1)-14(n) and a plurality of client devices 16(1)-16(n) via communication network(s) 18 and 20, respectively, although the surgical video analysis device 12, server devices 14(1)-14(n), and/or client devices 16(1)-16(n) may be coupled together via other topologies. Additionally, the network environment 10 may include other network devices such as one or more routers and/or switches, for example, which are well known in the art and thus will not be described herein. This technology provides a number of advantages including methods, non-transitory computer readable media, and surgical video analysis devices that automatically analyze video(s) of a surgical procedure by applying a neural network, for example, to surgical image data and contextual data associated with the surgical image data to efficiently and effectively identify and track objects in the video(s) to automatically generate a surgical report.

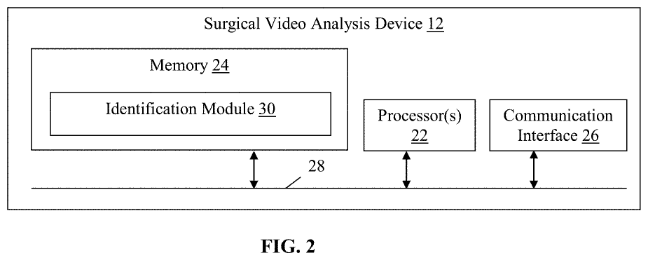

Referring to FIGS. 1-2, the surgical video analysis device 12 in this example includes processor(s) 22, a memory 24, and/or a communication interface 26, which are coupled together by a bus 28 or other communication link, although the surgical video analysis device 12 can include other types and/or numbers of elements in other configurations. The processor(s) 22 of the surgical video analysis device 12 may execute programmed instructions stored in the memory 24 for the any number of the functions described and illustrated herein. The processor(s) 22 of the surgical video analysis device 12 may include one or more CPUs or general purpose processors with one or more processing cores, for example, although other types of processor(s) can also be used.

The memory 24 of the surgical video analysis device 12 stores these programmed instructions for one or more aspects of the present technology as described and illustrated herein, although some or all of the programmed instructions could be stored elsewhere. A variety of different types of memory storage devices, such as random access memory (RAM), read only memory (ROM), hard disk, solid state drives, flash memory, or other computer readable medium which is read from and written to by a magnetic, optical, or other reading and writing system that is coupled to the processor(s) 22, can be used for the memory 24.

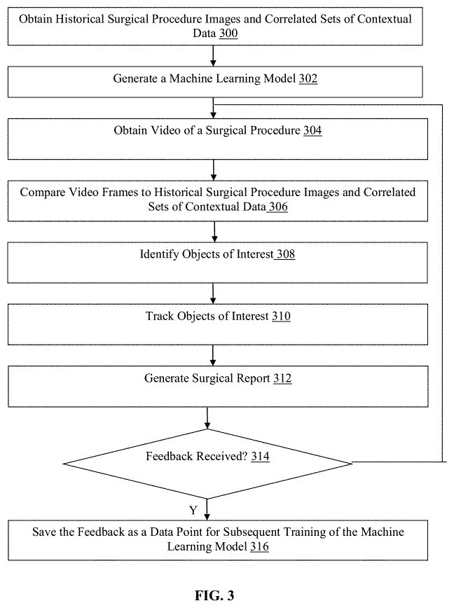

Accordingly, the memory 24 of the surgical video analysis device 12 can store application(s) that can include executable instructions that, when executed by the processor(s) 22, cause the surgical video analysis device 12 to perform actions, such as to transmit, receive, or otherwise process network messages, for example, and to perform other actions described and illustrated below with reference to FIG. 3. The application(s) can be implemented as modules or components of other application(s). Further, the application(s) can be implemented as operating system extensions, module, plugins, or the like.

Even further, the application(s) may be operative in a cloud-based computing environment. The application(s) can be executed within or as virtual machine(s) or virtual server(s) that may be managed in a cloud-based computing environment. Also, the application(s), and even the surgical video analysis device 12 itself, may be located in virtual server(s) running in a cloud-based computing environment rather than being tied to one or more specific physical network computing devices. Also, the application(s) may be running in one or more virtual machines (VMs) executing on the surgical video analysis device 12. Additionally, in one or more embodiments of this technology, virtual machine(s) running on the surgical video analysis device may be managed or supervised by a hypervisor.

In this particular example, the memory 24 of the surgical video analysis device 12 includes an identification module 30, although the memory 24 can include other policies, modules, databases, or applications, for example. The identification module 30 in this example is configured to train a machine learning model, such as an artificial or convolutional neural network, based on ingested, historical images of surgical procedures and sets of contextual data associated with the surgical procedures.

The identification module 30 is further configured to apply the neural network in one example to surgical video data and contextual data associated with the surgical video and automatically identify and track one or more objects in the surgical video as discussed in detail later with reference to FIG. 3. The one or more objects can include, by way of example, surgical instruments used in the surgical procedure, an anatomical structure, a fluid, or a structural abnormality in the surgical video. The tracked objects can be used to generate a surgical report related to the surgery that can include multiple pieces of information related to the surgery as described with respect to FIG. 3 below, among other items of information.

The communication interface 26 of the surgical video analysis device 12 operatively couples and communicates between the surgical video analysis device 12, the server devices 14(1)-14(n), and/or the client devices 16(1)-16(n), which are all coupled together by the communication network(s) 18 and 20, although other types and/or numbers of communication networks or systems with other types and/or numbers of connections and/or configurations to other devices and/or elements can also be used.

By way of example only, the communication network(s) 18 and 20 can include local area network(s) (LAN(s)) or wide area network(s) (WAN(s)), and can use TCP/IP over Ethernet and industry-standard protocols, although other types and/or numbers of protocols and/or communication networks can be used. The communication network(s) 18 and 20 in this example can employ any suitable interface mechanisms and network communication technologies including, for example, teletraffic in any suitable form (e.g., voice, modem, and the like), Public Switched Telephone Network (PSTNs), Ethernet-based Packet Data Networks (PDNs), combinations thereof, and the like.

The surgical video analysis device 12 can be a standalone device or integrated with one or more other devices or apparatuses, such as one or more of the server devices 14(1)-14(n), for example. In one particular example, the surgical video analysis device 12 can include or be hosted by one of the server devices 14(1)-14(n), and other arrangements are also possible.

Each of the server devices 14(1)-14(n) in this example includes processor(s), a memory, and a communication interface, which are coupled together by a bus or other communication link, although other numbers and/or types of network devices could be used. The server devices 14(1)-14(n) in this example host content associated with surgical procedures including surgical procedure data including images of surgical procedures and associated contextual information, such as surgical tools, anatomical structures, surgical maneuvers (e.g., type of incision), structural abnormalities, relationship between anatomical structures, etc.

Although the server devices 14(1)-14(n) are illustrated as single devices, one or more actions of the server devices 14(1)-14(n) may be distributed across one or more distinct network computing devices that together comprise one or more of the server devices 14(1)-14(n). Moreover, the server devices 14(1)-14(n) are not limited to a particular configuration. Thus, the server devices 14(1)-14(n) may contain a plurality of network devices that operate using a master/slave approach, whereby one of the network devices of the server devices 14(1)-14(n) operate to manage and/or otherwise coordinate operations of the other network devices.

The server devices 14(1)-14(n) may operate as a plurality of network devices within a cluster architecture, a peer-to peer architecture, virtual machines, or within a cloud architecture, for example. Thus, the technology disclosed herein is not to be construed as being limited to a single environment and other configurations and architectures are also envisaged.

The client devices 16(1)-16(n) in this example include any type of computing device that can interface with the surgical video analysis device 12 to submit data and/or receive GUI(s). Each of the client devices 16(1)-16(n) in this example includes a processor, a memory, and a communication interface, which are coupled together by a bus or other communication link, although other numbers and/or types of network devices could be used.

The client devices 16(1)-16(n) may run interface applications, such as standard web browsers or standalone client applications, which may provide an interface to communicate with the surgical video analysis device 12 via the communication network(s) 20. The client devices 16(1)-16(n) may further include a display device, such as a display screen or touchscreen, and/or an input device, such as a keyboard, for example. In one example, the client devices 16(1)-16(n) can be utilized by hospital staff to to facilitate an improved automatic surgical report generation, as described and illustrated herein, although other types of client devices utilized by other types of users can also be used in other examples. In one example, the client devices 16(1)-16(n) received data including patient information, such as name, date of birth, medical history, etc.; hospital information, such as hospital name or NHS number; temporal information, such as the date and time of the surgery; or surgical staff information, such as an identification of the operating surgeon, assistants, anesthetist, etc., for example. In other examples, this information is stored on one of the server devices 14(1)-14(n).

Although the exemplary network environment 10 with the surgical video analysis device 12, server devices 14(1)-14(n), client devices 16(1)-16(n), and communication network(s) 18 and 20 are described and illustrated herein, other types and/or numbers of systems, devices, components, and/or elements in other topologies can be used. It is to be understood that the systems of the examples described herein are for exemplary purposes, as many variations of the specific hardware and software used to implement the examples are possible, as will be appreciated by those skilled in the relevant art(s).

One or more of the devices depicted in the network environment 10, such as the surgical video analysis device 12, client devices 16(1)-16(n), or server devices 14(1)-14(n), for example, may be configured to operate as virtual instances on the same physical machine. In other words, one or more of the surgical video analysis device 12, client devices 16(1)-16(n), or server devices 14(1)-14(n) may operate on the same physical device rather than as separate devices communicating through communication network(s). Additionally, there may be more or fewer surgical video analysis devices, client devices, or server devices than illustrated in FIG. 1.

In addition, two or more computing systems or devices can be substituted for any one of the systems or devices in any example. Accordingly, principles and advantages of distributed processing, such as redundancy and replication also can be implemented, as desired, to increase the robustness and performance of the devices and systems of the examples. The examples may also be implemented on computer system(s) that extend across any suitable network using any suitable interface mechanisms and traffic technologies, including by way of example only wireless networks, cellular networks, PDNs, the Internet, intranets, and combinations thereof.

The examples may also be embodied as one or more non-transitory computer readable media (e.g., the memory 24) having instructions stored thereon for one or more aspects of the present technology as described and illustrated by way of the examples herein. The instructions in some examples include executable code that, when executed by one or more processors (e.g., the processor(s) 22), cause the processor(s) to carry out steps necessary to implement the methods of the examples of this technology that are described and illustrated herein.

An exemplary method of improved, automated surgical report generation will now be described with reference to FIG. 3. Referring more specifically to FIG. 3, a flowchart of an exemplary method for utilizing machine learning to identify and track multiple objects in a surgical video to automatically generate a surgical report is illustrated.

In step 300 in this example, the surgical video analysis device 12 obtains a training data set that includes surgical procedure images and a set of contextual data for the surgical procedures. The surgical procedure images and/or contextual data can be associated with historical surgical procedures and can be obtained from medical facilities hosting one or more of the server devices 14(1)-14(n) and/or other medical databases, for example, and other sources of one or more portions of the training data set can also be used. In another example, the historical set of surgical procedure images includes multispectral, hyperspectral, or molecular chemical imaging associated with the surgical procedure. In this example, the imaging is utilized as a contrast mechanism to assist in tissue critical structure segmentation as described below. These imaging techniques may also be employed to establish key points in the video of the surgery in order to assist in automated generation of a surgical report, as described in the examples herein. In one example, the historical surgical procedures are laparoscopic surgical procedures, although the disclosed methods can be employed for any surgical procedures. Additionally, the contextual data can include surgical instruments used in the surgical procedure, surgical techniques employed, an anatomical structure, a fluid, or a structural abnormality in the surgical video, patient demographic data, for example, although other types of contextual data can also be obtained in step 300. In one example, the contextual data can also include spatial, or intensity-based features for one or more objects in the historical set of surgical procedure images.

In step 302, the surgical video analysis device 12 generates or trains a machine learning model based on the training data set including the surgical procedure images and correlated sets of contextual data obtained in step 300. In one example, the machine learning model is a neural network, such as an artificial or convolutional neural network, although other types of neural networks or machine learning models can also be used in other examples. In one example, the neural network is a fully convolutional neural network. In this example, the surgical video analysis device 12 can generate the machine learning model by training the neural network using the surgical procedure images and correlated sets of contextual data obtained in step 300.

In step 304, the surgical video analysis device 12 obtains a new video(s) associated with a surgical procedure comprising a plurality of frames that provide images of the surgical procedure. The video(s) can be obtained from one or more of the server devices 14(1)-14(n) and/or one of the client devices 16(1)-16(n), for example. In one example, the video(s) is an intra-operative video of a laparoscopic surgical procedure, although this technology may be employed with other videos of other types of surgical procedures. The surgical video analysis device may also receive multispectral, hyperspectral, or molecular chemical imaging data associated with the video.

In step 306, the surgical analysis device 12 applies the machine learning model to the plurality of frames of the videos(s) to compare the plurality of frames of the obtained video to the historical set of surgical procedure images and correlated sets of contextual data obtained in step 300. In step 308, the surgical video analysis device 12 identifies one or more objects of interest or regions of interest appearing in at least a subset of the plurality of frames based on the comparison of the video to the historical set of surgical procedure images and the associated contextual information. The surgical video analysis device 12 advantageously identifies multiple objects in the surgical video. The objects, or regions, of interest can include, for example, one or more of a surgical instruments used in the surgical procedure, an anatomical structure, a fluid, or a structural abnormality. In one example, the objects in surgery video are identified using the fully convolutional network (FCN), which learns representations and make the decisions based on local spatial features. In one example, the UNet architecture as described in Ronneberger, O., et al., “U-net: Convolutional networks for biomedical image segmentation,” International Conference on Medical image computing and computer-assisted intervention (pp. 234-241), Springer, Cham. (October 2015), the disclosure of which is incorporated herein by reference in its entirety, it utilized for the identification. The advantage of this structure is that it was first designed for medical image segmentation, which makes it inherently suitable for surgery video classification work. Another advantage is UNet has the build-in data augmentation method, which allows utilizing small training sets (<100 images). In yet another example, the historical set of surgical procedure images includes multispectral, hyperspectral, or molecular chemical imaging, which may be employed as contrast mechanism to assist in tissue critical structure segmentation.

In step 310, the surgical video analysis device 12 tracks the identified one or more objects of interest across the at least the subset of the plurality of frames. The objects may be tracked, for example, to identify the surgical technique employed, changes in the structural anatomy, fluid flow in the video, etc. In one example, the objects are tracked based on an intensity based tracking method or a feature based tracking method, such as, by way of example only, Meanshift Tracking, Kalman Filters, and Optical Flow Tracking. The tracked one or more objects comprise one or more of a surgical instruments used in the surgical procedure, an anatomical structure, a fluid, or a structural abnormality visible in the video. The surgical video analysis device 12 not only spatially identifies the structures and surgical tools, but also learns their dynamic relationship during the operation using temporal tracking. Therefore, the surgical video analysis device 12 can generate contents that directly describe the complete operative procedure as described in further detail below. In one example, the historical set of surgical procedure images includes multispectral, hyperspectral, or molecular chemical imaging associated with the surgical procedure that may be employed establish key points in the video of the surgery in order to assist in automated generation of a surgical report.

Advantageously, analyzing digital surgical videos and contextual data automatically using a machine learning model provides a practical application of this technology in the form of earlier, automated, consistent, and objective identification and tracking of multiple objects in the video, and solves a technical problem in the video analysis art. In examples in which a neural network is used for the machine learning model, the neural network can leverage certain features of the obtained videos(s), such as spatial features or intensities in the video(s), for example and particular portions of the obtained contextual data, which is merged with the historical videos and set of contextual data used to train the neural network, to identify and track multiple objects in the surgical video. Other methods of applying the machine learning model and/or automatically identifying and tracking objects can also be used in other examples.

Examples of tracked objects in the video(s) can include the following:

(1) Identified structures and fluids: the major anatomical structures encountered are identified and analyzed quantitatively by calculating their semantic descriptors (e.g. shape, color and textures). By comparing descriptors with features in the pre-trained classifier, surgical video analysis device 12 can determine if the structures in the video are as expected. The FCN can also identify and quantitatively measure fluid during the surgery. One example would be to indicate a significant blood loss by measuring the blood coverage on the video frames.

(2) The relationship among the structures: The information from the identification of multiple structures is combined into representations, which spatially clarify the perception of static relationships and can highlight the locations and types of structural abnormalities shown in the video. The temporal tracking results can further identify the dynamic relationship with the surgical instruments and maneuvers, exposing new tissue relationships and structures.

(3) The identified surgical instruments: The FCN can identify and track the surgical instruments during the operation. The tracking results should indicate which surgical instrument are used, how they are used, and anatomically where they are used. These are merely examples and are not intended to be limiting.

In step 312, the surgical video analysis device 12 automatically generates a surgical report based on the tracked one or more objects. The surgical report includes an identification of the tracked objects and information related to the tracked objects, including for example, the information of the above examples. The information determined using the machine learning model can, for example, be inserted into a surgical report template. The surgical video analysis device 12 provides the intra-operative details on the generated report. The intra-operative details incorporated in the generated report may include surgical tool movement, major structures encountered, unexpected complications found, or any tissue removed. In addition, the operative data can be merged with the patient specific information and information generated by the operating surgeon. In one example, the surgical video analysis device 12 automatically links the identified one or more objects, and associated contextual information obtained using the machine learning model, to the subset of the plurality of frames over which the identified one or more objects are tracked. The information can then be stored on a picture archiving and communication system (PACS), which allows for easy data access for future use, for example, for additional surgeries for the patient, clinical research, insurance purposes, evaluating surgical performance, etc. In another example, the surgical video analysis device 12 automatically associates one or more general items of data related to the surgical procedure to the generated surgical report that may be included in the template, such as hospital information, temporal information (date and time of the surgery), or surgical staff information.

In step 314, the surgical video analysis device 12 optionally determines whether any feedback is received with respect to the tracked items identified in the surgical report generated in step 312 that can be used to further train the machine learning model.

If the surgical video analysis device 12 determines that feedback is received, then the Yes branch is taken step 316, and the feedback data, along with associated surgical video(s) and contextual data, are saved as a data point for future training data sets that can be used to further train or update the machine learning model, as described earlier with reference to step 302. Subsequent to saving the feedback as a data point in step 316, or if the surgical video analysis device 12 determines in step 314 that feedback is not received and the No branch is taken, then the surgical video analysis device 12 proceeds back to step 304 and again obtains video(s) of a surgical procedure.

### EXAMPLES

### Example 1—Tracking Multiple Regions of Interest

A multiple region of interest (ROI) tracking framework was developed in Matlab based on dense optical flow tracking using the Farneback method as disclosed in Farneback, G., “Very High Accuracy Velocity Estimation Using Orientation Tensors, Parametric Motion and Simultaneous Segmentation of the Motion Field,” Proc. 8th International Conference on Computer Vision. Volume 1., IEEE Computer Society Press (2001), the disclosure of which is incorporated herein by reference in its entirety. The framework was tested on various endoscopic Storz videos from a surgery dataset. The Storz video was re-processed to better simulate tracking condition under MCI-E Gen2 Camera. The resolution of the Storz video was downsampled from 1920×1080 to 640×360 and the frame rate was resampled from 27 FPS to 9 FPS. The tracking framework was advantageously able to determine shape and appearance change and large and fast motions within the ROI.

### Example 2—Training Using U-Net

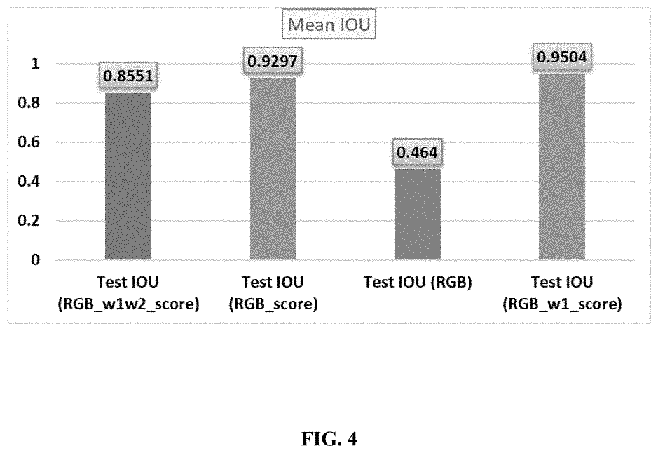

A video containing 100 frames was analyzed using U-Net. The first 30 frames in the video (Elastic Deformation Data Augmentation used, hence total 60 frames for training) were used for training and frames 31 to 100 (70) frames from the video were used for testing. As shown in FIG. 4, testing Performance using R, G, B, w1, score provided better performance than just R, G, B (or) R, G, B, w1, w2, score (or) R, G, B, score. Using the R, G, B, score, provided the following mean IOU values: Final 30 frames: 0.9069; Final 70 frames: 0.9297. False positives increase as the frame number increases. Hence, using previous frame information could improve the results. The w1 and w2 provide redundant information (as the data samples are correlated to score image) and hence less performance (score=w1/w2). Score image information provides a significant increase in performance of the network when compared to just R, G, B.

With this technology, multiple objects in a surgical video can be identified and tracked more efficiently based on an automated analysis of videos(s) of a surgical procedure, and a surgical report can be generated, without requiring any input from the surgeon. This technology utilizes videos analysis and a machine learning model, such as a neural network, to advantageously generate a more consistent, objective surgical report automatically and, in the context of surgical procedures, earlier in the process.

In the above detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be used, and other changes may be made, without departing from the spirit or scope of the subject matter presented herein. It will be readily understood that various features of the present disclosure, as generally described herein, and illustrated in the Figures, can be arranged, substituted, combined, separated, and designed in a wide variety of different configurations, all of which are explicitly contemplated herein.

The present disclosure is not to be limited in terms of the particular embodiments described in this application, which are intended as illustrations of various features. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, compositions or biological systems, which can, of course, vary. It is also to be understood that the terminology used herein is for the purpose of describing particular embodiments only, and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (for example, bodies of the appended claims) are generally intended as “open” terms (for example, the term “including” should be interpreted as “including but not limited to,” the term “having” should be interpreted as “having at least,” the term “includes” should be interpreted as “includes but is not limited to,” et cetera). While various compositions, methods, and devices are described in terms of “comprising” various components or steps (interpreted as meaning “including, but not limited to”), the compositions, methods, and devices can also “consist essentially of” or “consist of” the various components and steps, and such terminology should be interpreted as defining essentially closed-member groups. It will be further understood by those within the art that if a specific number of an introduced claim recitation is intended, such an intent will be explicitly recited in the claim, and in the absence of such recitation no such intent is present.

For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (for example, “a” and/or “an” should be interpreted to mean “at least one” or “one or more”); the same holds true for the use of definite articles used to introduce claim recitations.

In addition, even if a specific number of an introduced claim recitation is explicitly recited, those skilled in the art will recognize that such recitation should be interpreted to mean at least the recited number (for example, the bare recitation of “two recitations,” without other modifiers, means at least two recitations, or two or more recitations). Furthermore, in those instances where a convention analogous to “at least one of A, B, and C, et cetera” is used, in general such a construction is intended in the sense one having skill in the art would understand the convention (for example, “a system having at least one of A, B, and C” would include but not be limited to systems that have A alone, B alone, C alone, A and B together, A and C together, B and C together, and/or A, B, and C together, et cetera). In those instances where a convention analogous to “at least one of A, B, or C, et cetera” is used, in general such a construction is intended in the sense one having skill in the art would understand the convention (for example, “a system having at least one of A, B, or C” would include but not be limited to systems that have A alone, B alone, C alone, A and B together, A and C together, B and C together, and/or A, B, and C together, et cetera). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, et cetera. As a non-limiting example, each range discussed herein can be readily broken down into a lower third, middle third and upper third, et cetera. As will also be understood by one skilled in the art all language such as “up to,” “at least,” and the like include the number recited and refer to ranges that can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

Various of the above-disclosed and other features and functions, or alternatives thereof, may be combined into many other different systems or applications. Various presently unforeseen or unanticipated alternatives, modifications, variations or improvements therein may be subsequently made by those skilled in the art, each of which is also intended to be encompassed by the disclosed embodiments.

What is claimed is:

1. A method for improved, automated surgical report generation, the method comprising:
obtaining, by a surgical video analysis device, a video associated with a surgical procedure comprising a plurality of frames;
comparing, by the surgical video analysis device, the plurality of frames of the obtained video to a historical set of surgical procedure images, wherein the historical set of surgical procedure images are associated with contextual information;
identifying, by the surgical video analysis device, one or more objects of interest in at least a subset of the plurality of frames based on the comparison and the associated contextual information;
tracking, by the surgical video analysis device, the identified one or more objects of interest across the at least the subset of the plurality of frames;
generating, by the surgical video analysis device, a surgical report based on tracked one or more objects.

2. The method of claim 1 further comprising applying, by the surgical video analysis device, a machine learning model to identify the one or more objects of interest in the at least the subset of the plurality of frames.

3. The method of claim 2, wherein the machine learning model comprises a fully convolutional neural network.

4. The method of claim 2, wherein the associated contextual information comprises spatial features for one or more objects in the historical set of surgical procedure images.

5. The method of claim 1, wherein the historical set of surgical procedure images comprise multispectral, hyperspectral, or molecular chemical imaging data.

6. The method of claim 1, wherein the identified one or more objects of interest are tracked based on an intensity based tracking method or a feature based tracking method.

7. The method of claim 1, wherein the tracked one or more objects comprise one or more of a surgical instruments used in the surgical procedure, an anatomical structure, a fluid, or a structural abnormality.

8. The method of claim 1, wherein the generated surgical report comprises an identification of tracked one or more objects.

9. The method of claim 8 further comprising:
linking, by the surgical video analysis device, the identified one or more objects to the subset of the plurality of frames over which the identified one or more objects are tracked.

10. The method of claim 1 further comprising:
associating, by the surgical video analysis device, one or more items of data related to the surgical procedure to the generated surgical report.

11. The method of claim 8, wherein the one or more items of data comprise patient information, hospital information, temporal information, or surgical staff information.

12. A surgical video analysis device, comprising memory comprising programmed instructions stored thereon and one or more processors configured to execute the stored programmed instructions to:
obtain a video associated with a surgical procedure comprising a plurality of frames;
compare the plurality of frames of the obtained video to a historical set of surgical procedure images, wherein the historical set of surgical procedure images are associated with contextual information;
identify one or more objects of interest in at least a subset of the plurality of frames based on the comparison and the associated contextual information;
track the identified one or more objects of interest across the at least the subset of the plurality of frames;
generate a surgical report based on tracked one or more objects.

13. The device of claim 12, wherein the processors are further configured to execute the stored programmed instructions to apply a machine learning model to identify the one or more objects of interest in the at least the subset of the plurality of frames.

14. The device of claim 13, wherein the machine learning model comprises a fully convolutional neural network.

15. The device of claim 13, wherein the associated contextual information comprises spatial features for one or more objects in the historical set of surgical procedure images.

16. The device of claim 12, wherein the historical set of surgical procedure images comprise multispectral, hyperspectral, or molecular chemical imaging data.

17. The device of claim 12, wherein the identified one or more objects of interest are tracked based on an intensity based tracking method or a feature based tracking method.

18. The device of claim 12, wherein the tracked one or more objects comprise one or more of a surgical instruments used in the surgical procedure, an anatomical structure, a fluid, or a structural abnormality.

19. The device of claim 12, wherein the generated surgical report comprises an identification of tracked one or more objects.

20. The device of claim 19, wherein the processors are further configured to execute the stored programmed instructions to link the identified one or more objects to the subset of the plurality of frames over which the identified one or more objects are tracked.

21. The device of claim 12, wherein the processors are further configured to execute the stored programmed instructions to associate one or more items of data related to the surgical procedure to the generated surgical report.

22. The device of claim 19, wherein the one or more items of data comprise patient information, hospital information, temporal information, or surgical staff information.

23. A non-transitory machine readable medium having stored thereon instructions for improved, automated surgical report generation comprising executable code that, when executed by one or more processors, causes the processors to:
obtain a video associated with a surgical procedure comprising a plurality of frames;
compare the plurality of frames of the obtained video to a historical set of surgical procedure images, wherein the historical set of surgical procedure images are associated with contextual information;
identify one or more objects of interest in at least a subset of the plurality of frames based on the comparison and the associated contextual information;
track the identified one or more objects of interest across the at least the subset of the plurality of frames;
generate a surgical report based on tracked one or more objects.

24. The non-transitory machine readable medium of claim 23, wherein the executable code, when executed by the processors, further causes the processors to apply a machine learning model to identify the one or more objects of interest in the at least the subset of the plurality of frames.

25. The non-transitory machine readable medium of claim 24, wherein the machine learning model comprises a fully convolutional neural network.

26. The non-transitory machine readable medium of claim 24, wherein the associated contextual information comprises spatial features for one or more objects in the historical set of surgical procedure images.

27. The non-transitory machine readable medium of claim 23, wherein the historical set of surgical procedure images comprise multispectral, hyperspectral, or molecular chemical imaging data.

28. The non-transitory machine readable medium of claim 23, wherein the identified one or more objects of interest are tracked based on an intensity based tracking method or a feature based tracking method.

29. The non-transitory machine readable medium of claim 23, wherein the tracked one or more objects comprise one or more of a surgical instruments used in the surgical procedure, an anatomical structure, a fluid, or a structural abnormality.

30. The non-transitory machine readable medium of claim 23, wherein the generated surgical report comprises an identification of tracked one or more objects.

31. The non-transitory machine readable medium of claim 30, wherein the executable code, when executed by the processors, further causes the processors to link the identified one or more objects to the subset of the plurality of frames over which the identified one or more objects are tracked.

32. The non-transitory machine readable medium of claim 23, wherein the executable code, when executed by the processors, further causes the processors to associate one or more items of data related to the surgical procedure to the generated surgical report.

33. The non-transitory machine readable medium of claim 30, wherein the one or more items of data comprise patient information, hospital information, temporal information, or surgical staff information.
